(19) United States
(12) Patent Application Publication
(10) Pub. No.: US 2014/0303700 A1
(43) Pub. Date: Oct. 9, 2014

(54) COLD THERAPY SHOES

(71) Applicant: David R. Hinkle, Austin, TX (US)

(72) Inventor: David R. Hinkle, Austin, TX (US)

(21) Appl. No.: 14/231,270

(22) Filed: Mar. 31, 2014

Related U.S. Application Data
(60) Provisional application No. 61/808,853, filed on Apr. 5, 2013.

(51) Int. Cl. A61F 7/02; A43B 7/02; A43B 7/00

(52) U.S. Cl. CPC A61F 7/02; A43B 7/005; A43B 7/02
USPC 607/111; 12/142 R

(57) ABSTRACT

A method and footwear for providing therapeutic elements, the footwear includes a sole, and a therapeutic element, wherein the therapeutic element is formed in the sole. The method includes providing a footwear mould, injecting a footwear material into the footwear mould, removing a footwear from the footwear mould, and fusing a therapeutic element to the footwear.

### CROSS-REFERENCE TO PRIOR APPLICATIONS

This application claims priority to and the benefit thereof from U.S. Provisional Patent Application No. 61/808,853, filed Apr. 5, 2013, and titled “COLD THERAPY SHOES,” the entirety of which is hereby incorporated herein by reference.

### FIELD OF THE DISCLOSURE

The present disclosure relates generally to a method and apparatus for providing therapy, and more specifically it relates to a method and an apparatus for providing therapy to a foot.

### BACKGROUND OF THE DISCLOSURE

Footwear comes in various types, styles, shapes, and sizes. Footwear such as, for example, sandals, flip-flops, sneakers, and the like, are commonly worn every day when e.g., commuting, shopping, running errands, and so on.

It is known that applying ice to an injured area can be effective for reducing pains associated with strains, overuse injuries, and bruises. It is also known that heat can help treat an injured area by increasing blood flow to a heated area which in turn brings along oxygen and nutrients that can help with the healing process. Heat can also help relax muscles, which can decrease pain.

While therapeutic products that specifically provide cold or heat sensation are known, these often exist as stand-alone products and not as integral parts of normal footwear that can be worn every day. These products do not blend in well with ordinary clothing. Instead, the products noticeably stand out as therapeutic devices.

Furthermore, these products are often incompatible with actual use in e.g., walking, jogging, and so on. There exists an unfulfilled need for footwear that can provide therapeutic elements (e.g., heat, cold, massage, and the like) and that can be worn during everyday use.

### SUMMARY OF THE DISCLOSURE

Accordingly, one aspect of the present disclosure provides footwear for providing therapeutic elements. The footwear includes a sole, and a therapeutic element, wherein the therapeutic element is formed in the sole.

The footwear may include a strap which may further include a Y-shaped or a single strap.

The therapeutic element may provide pressure and cold therapy to an area of a foot in contact with the therapeutic element.

Alternatively, the therapeutic element may provide pressure and heat therapy to an area of a foot in contact with the therapeutic element.

The therapeutic element may also include a material that carries out an endothermic or exothermic reaction.

The footwear may further include a barrier layer that protects a wearer's foot from direct contact and exposure to the therapeutic element.

The therapeutic element may be detachable from the sole.

The footwear may include a sock, a sneaker, a slipper, a sandal, a tennis shoe, a running shoe, an athletic shoe, a boot, a dress shoe, a high heel shoe, a rain shoe, or a flip-flop.

According to another aspect of the present disclosure, a method of manufacturing a footwear having an integrated therapeutic element is provided. The method includes providing a footwear mould, injecting a footwear material into the footwear mould, removing a footwear from the footwear mould, and fusing a therapeutic element to the footwear.

The footwear may include at least one of polyurethane (PU), thermoplastic rubber (TPR), polyvinyl chloride, ethylene-vinyl acetate (EVA), rubber, foam, and plastic.

The therapeutic element may include a material that provides pressure and cold therapy to an area of a foot in contact with the therapeutic element.

The therapeutic element may include a material that provides pressure and heat therapy to an area of a foot in contact with the therapeutic element.

The therapeutic element may further include a material that carries out an endothermic reaction or exothermic reaction. The therapeutic element may further include a gel.

The footwear may be formed with a barrier layer that includes a cavity to receive a therapeutic material. The therapeutic element may be formed to be detachable from the footwear.

### BRIEF DESCRIPTION OF THE DRAWINGS

The attached drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced. In the drawings:

FIG. 1 shows an example of a shoe having an integrated therapeutic element in the ball area according to the principles of the disclosure.

FIG. 2 shows a side view of the shoe in FIG. 1.

FIG. 3 shows an example of a shoe having an integrated therapeutic element in the heel area according to the principles of the disclosure.

FIG. 4 shows a side view of the she in FIG. 3.

FIG. 5 shows an example of a method of manufacturing a shoe having an integrated therapeutic element according to the principles of the disclosure.

### DETAILED DESCRIPTION OF THE DISCLOSURE

The embodiments of the disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure, which is defined solely by the appended claims and applicable law. Moreover, it is noted that like reference numerals represent similar parts throughout the several views of the drawings.

The terms “including,” “comprising” and variations thereof, as used in this disclosure, mean “including, but not limited to,” unless expressly specified otherwise.

The terms “a,” “an,” and “the,” as used in this disclosure, means “one or more”, unless expressly specified otherwise.

Although process steps, method steps, algorithms, or the like, may be described in a sequential order, such processes, methods and algorithms may be configured to work in alternate orders. In other words, any sequence or order of steps that may be described does not necessarily indicate a requirement that the steps be performed in that order. The steps of the processes, methods or algorithms described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device may be alternatively embodied by one or more other devices which are not explicitly described as having such functionality or features.

Footwear is described herein that incorporates a therapeutic element in one or more portions of the footwear. The therapeutic element may be positioned in a heel area and/or a ball area of the footwear. The therapeutic element may be positioned in other areas of the footwear, other than the heel or ball areas of the footwear. While the examples described herein are directed to flip-flops, the footwear may include for example, a sock, a sneaker, a slipper, a sandal, a tennis shoe, a running shoe, an athletic shoe, a boot, a dress shoe, a high heel shoe, a rain shoe, and so on. The therapeutic element is constructed to apply cold, hot, and/or massage therapy to a specific area of a user's foot.

The therapeutic element may include a gel, which may include propylene glycol, hydroxyethlyl cellulose, vinyl-coated silica, or the like, that retains cold temperatures when placed in, e.g., a freezer. The gel should be flexible enough to conform to a foot's contours at low temperatures (e.g., below 32° F.).

The therapeutic element may include, e.g., a supersaturated solution of sodium acetate (CH₃COONa) in water, a high specific heat capacity solution, or the like, which will allow for the element to be repeatedly heated and used.

The therapeutic element may include a material that carries out an endothermic reaction or process, and/or a material that carries out an exothermic reaction or process. For instance, the material may include separate areas that include compounds such as, for example, thionyl chloride (SOCl₂) and cobalt (II) sulfate heptahydrate, water and ammonium nitrate, or the like, which when mixed cause an endothermic reaction to occur. The areas may be separated by a membrane that, when broken, allows the different compounds to mix.

The material may include, for example, sodium acetate and a metal disc activator, as is known in the art, which when activated causes the solution to go from liquid to solid, releasing heat in the process.

As one skilled in the art will appreciate, the therapeutic element may include any other material that will retain cold (or hot) temperatures for extended periods of time and that will be flexible enough to conform to the contours of a user's foot.

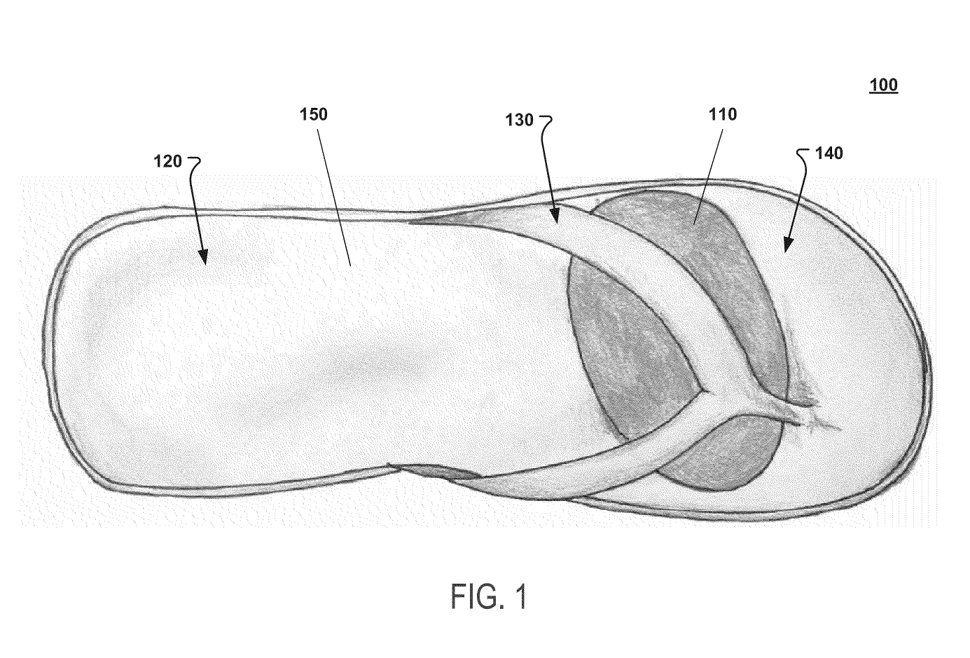

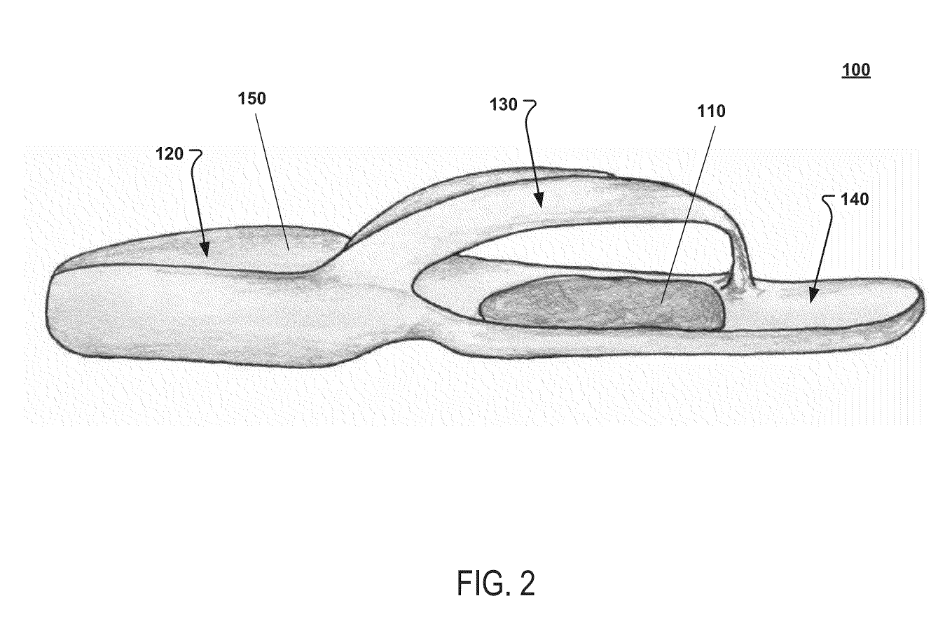

FIGS. 1 and 2 show top and side views of an example of footwear 100 that is constructed according to the principles of the disclosure. The footwear is configured to provide cold, heat and/or massage therapy to a user's foot. The footwear 100 may include a Y-shaped strap 130 and a sole 150. The sole 150 may be held loosely on the foot by the Y-shaped strap 130 that passes between the first and second toes and around either sides of the foot. Alternatively, the strap may include a single strap over the front of the foot rather than a Y-shaped strap. The sole 150 and the Y-shaped strap 130 may be made from a material, such as, e.g., rubber, foam, plastic, leather, suede, fabric, carbon fiber, metal, and the like.

Referring to FIGS. 1 and 2 concurrently, the footwear 100 may include a therapeutic element 110 located in an area 140 where the ball of the foot would be located when wearing the footwear 100. The therapeutic element 110 may be an integrated portion of the footwear 100. Alternatively, the therapeutic element 110 may be attachable (or detachable) to the sole 150 via e.g., an adhesive, a hook-and-loop fastener, or the like.

The sole 150 may include e.g., a recess, an indented portion, or the like, which may receive and house the therapeutic element 110. The therapeutic element 110 may be configured to extend above the sole 150 so that it appears to be above the sole 150 when viewed from a side perspective. Alternatively, the therapeutic element 110 may be configured to be substantially flush with the top surface of the sole 150. The therapeutic element 110 may also be configured to extend below the top surface of the sole 150.

The footwear 100 may be placed in, e.g., a freezer to cool the therapeutic element 110 to a low temperature (e.g., below 32° F.) prior to wearing. Once cooled to the low temperature, a user can wear the footwear 100 and have cold therapy applied to the foot without being required to sit down and apply an ice pack to the foot. The therapeutic element 110 may include any material (e.g., liquid, gel, or the like) that can be cooled and that will retain the cold temperature for an extended period of time when contacted by a user's foot.

When in its cold state, the therapeutic element 110 may provide light pressure and cold therapy to the area of the foot in contact with the therapeutic element. The therapeutic element should conform to the contour of the user's foot to provide a massaging action, so that to the area of the foot in contact with the therapeutic element 110 may receive therapeutic pressure from the contact area of the element as the wearer walks. The contact portion of the therapeutic element 110 may include a barrier layer, which may include a wear resistant flexible material, such as, for example, rubber, foam, fabric, leather, suede, polyurethane, carbon fiber, and the like. The material may protect the user's foot from directly contacting the material in the therapeutic element 110, which could be extremely cold or extremely hot.

Alternatively, the footwear 100 may be placed in a heating device, such as, e.g., a microwave, a stove, a hot-pot of water, or the like, to heat the therapeutic element 110 to a high temperature (e.g., greater than 100° F.). Once heated to the high temperature, the user can wear the footwear 100 and have heat therapy applied to the foot without being required to sit down and apply a heat pack to the foot. The therapeutic element 110 may include any material (e.g., liquid, gel, or the like) that can be heated and that will retain a high temperature for an extended period of time when contacted by the user's foot. In its heated state, the therapeutic element 110 may provide light pressure and heat therapy to the area of the foot in contact with the element 110.

While FIG. 1 shows the therapeutic element 110 located only in the ball of the foot area, the element 110 may be located in any one or more portions of the footwear 100.

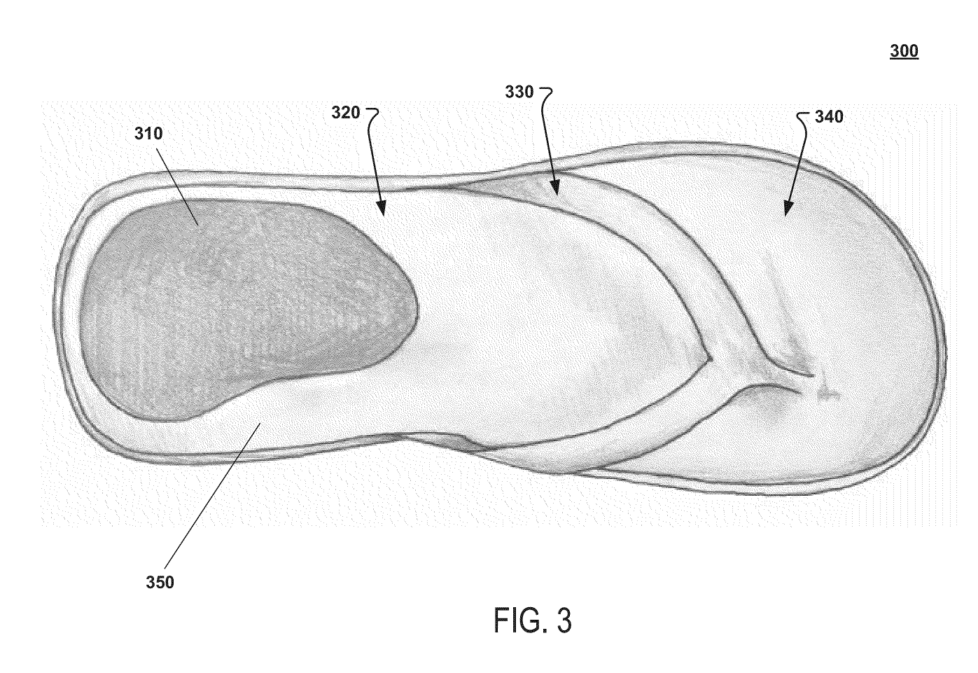

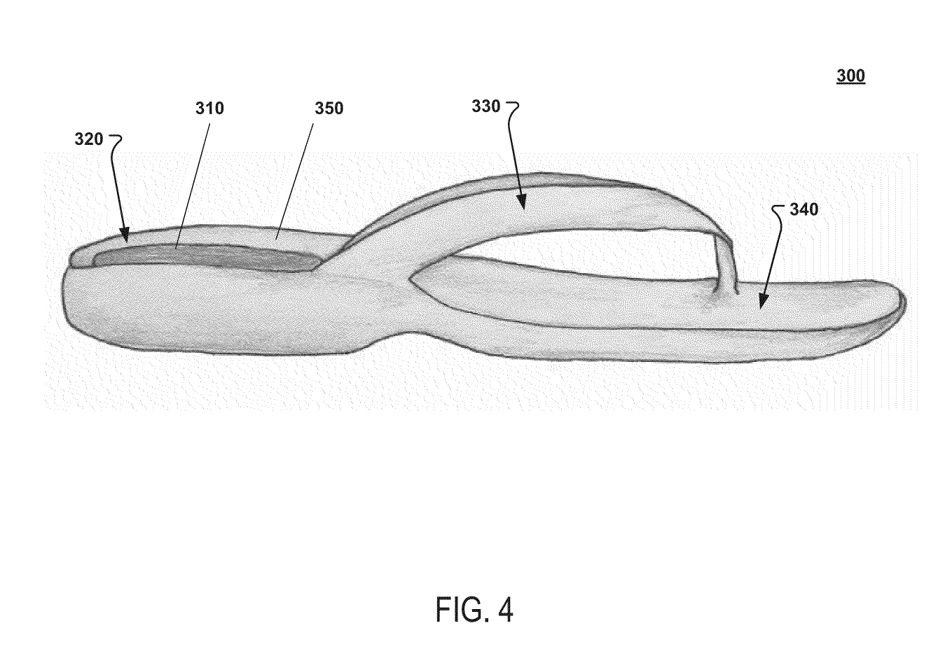

FIGS. 3 and 4 show top and side views of an example of footwear 300, constructed according to principles of the disclosure. The footwear 300 includes similar components to the footwear 100 shown in FIGS. 1 and 2. The footwear 300 further includes a therapeutic element 310 provided in the heel area 320 of the footwear 300. Alternatively, the therapeutic element 310 may be attached to a ball area 340 or any other area of the footwear 300.

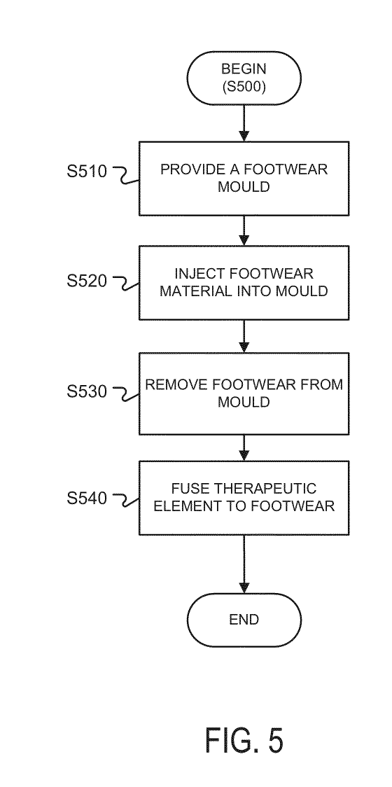

FIG. 5 shows an example of a method of manufacturing a footwear 100 (or 300) having an integrated therapeutic element 110 (and/or 310). The method includes providing a footwear mould (Step 510), injecting a footwear material into the footwear mould (Step 520), removing a footwear from the footwear mould (Step 530), and fusing a therapeutic element to the footwear (Step 540).

The footwear mould may include e.g., a casing that is made from e.g., metal, plastic, or the like. The footwear material may include e.g., polyurethane (PU), thermoplastic rubber (TPR), polyvinyl chloride (PVC), ethylene-vinyl acetate (EVA), rubber, foam, plastic, or the like. The footwear molding process may include e.g., blow molding, compaction plus sintering, compression molding, expandable bead molding, extrusion molding, foam molding, injection molding, laminating, rotational molding, transfer molding, thermoforming, and the like. The therapeutic element may be fused into the mould using e.g., adhesive, heating process, stitching, direct injection, any fastening mechanism, and the like. Alternatively, the sole 150 and therapeutic element may be integrally and substantially simultaneously formed in the mould. In this regard, the therapeutic element 110 may include a cavity that may be filled with a therapeutic element material (e.g., a gel).

Alternatively, the footwear material may be formed using known, existing processes, other than moulding, such as, for example, processes for making the footwear 100 out of leather, suede, fabric, carbon fiber, metal, or the like.

The therapeutic element 110 may include e.g., a gel, which may include propylene glycol, hydroxyethlyl cellulose, vinyl-coated silica, or the like, that retains cold temperatures when placed in, e.g., a freezer. The gel should be flexible enough to conform to a foot's contours at low temperatures (e.g., below 32° F.). The gel may be attached to any part of the sole via e.g., a tape, an adhesion, any fastening mechanisms, and the like. The gel may further be detachable from the sole for e.g., replacement, modification, and so on.

The therapeutic element may also include, e.g., a supersaturated solution of sodium acetate (CH₃COONa) in water, a high specific heat capacity solution, or the like, which will allow for the element to be repeatedly heated and used.

The therapeutic element may further include a material that carries out an endothermic reaction or process, and/or a material that carries out an exothermic reaction or process.

While the invention has been described in terms of exemplary embodiments, those skilled in the art will recognize that the invention can be practiced with modifications in the spirit and scope of the appended claims. These examples given above are merely illustrative and are not meant to be an exhaustive list of all possible designs, embodiments, applications or modifications of the invention.

What is claimed is:

1. A footwear comprising:
a sole; and
a therapeutic element,
wherein the therapeutic element is formed, in the sole.

2. The footwear according to claim 1 further comprising a strap.

3. The footwear according to claim 2 wherein the strap further comprises a Y-shaped strap.

4. The footwear according to claim 2 wherein the strap further comprises a single strap.

5. The footwear according to claim 1 wherein the therapeutic element provides pressure and cold therapy to an area of a foot in contact with the therapeutic element.

6. The footwear according to claim 1 wherein the therapeutic element provides pressure and heat therapy to an area of a foot in contact with the therapeutic element.

7. The footwear according to claim 1 wherein the therapeutic element comprises a material that carries out an endothermic reaction.

8. The footwear according to claim 1 wherein the therapeutic element comprises a material that carries out an exothermic reaction.

9. The footwear according to claim 1 further comprising:
a barrier layer that protects a wearer's foot from direct contact and exposure to the therapeutic element.

10. The footwear according to claim 1 wherein the therapeutic element is detachable from the sole.

11. The footwear according to claim 1 wherein the footwear comprises a sock, a sneaker, a slipper, a sandal, a tennis shoe, a running shoe, an athletic shoe, a boot, a dress shoe, a high heel shoe, a rain shoe, or a flip-flop.

12. The method of manufacturing a footwear having an integrated therapeutic element comprising:
providing a footwear mould;
injecting a footwear material into the footwear mould;
removing a footwear from the footwear mould; and
fusing a therapeutic element to the footwear.

13. The method according to claim 12 wherein the footwear material further comprises at least one of:
polyurethane (PU);
thermoplastic rubber (TPR);
polyvinyl chloride;
ethylene-vinyl acetate (EVA);
rubber;
foam; and
plastic.

14. The method according to claim 12 wherein the therapeutic element comprises a material that provides pressure and cold therapy to an area of a foot in contact with the therapeutic element.

15. The method according to claim 12 wherein the therapeutic element comprises a material that provides pressure and heat therapy to an area of a foot in contact with the therapeutic element.

16. The method according to claim 12 wherein the therapeutic element comprises a material that carries out an endothermic reaction.

17. The method according to claim 12 wherein the therapeutic element comprises a material that carries out an exothermic reaction.

18. The method according to claim 12 wherein the therapeutic element comprises a gel.

19. The footwear according to claim 12 wherein the footwear is formed with a harrier layer that includes a cavity to receive a therapeutic material.

20. The method according to claim 12 wherein the therapeutic element is formed to be detachable from the footwear.
